(19) United States
(12) Patent Application Publication
(10) Pub. No.: US 2010/0076564 A1
(43) Pub. Date: Mar. 25, 2010

(54) Tibial tuberosity advancement implant

(76) Inventors: Eric M. Schilling, Muhleim (DE); Harold M. Wotton, III, Woodstock, CT (US)

Correspondence Address: Joseph S. Iandiorio, 260 Bear Hill Road, Waltham, MA, 02451-1018, US

(21) Appl. No.: 12/284,483

(22) Filed: Sep. 23, 2008

(51) Int. Cl. A61F 2/38; A61B 17/58

(52) U.S. Cl. 623/20.14; 606/88

(57) ABSTRACT

A tibial tuberosity advancement implant with first and second arms pivotally joined at an apex. Tabs including anchor apertures extend from each arm for attachment of the arms in a traverse osteotomyized tibia. A selection of various width keys are each insertable between the arms for setting the distance between the arms and the width of the osteotomyized tibia.

### FIELD OF THE INVENTION

The subject invention relates to implants including a tibial tuberosity advancement implant.

### BACKGROUND OF THE INVENTION

The veterinary surgical procedure known as tibial tuberosity advancement is used to correct a rupture of the CrCL in the knee of a dog. “See Tibial Tuberosity Advancement” by Dr. Jeff Mayo incorporated herein by this reference. Typically, a tibial tuberosity advancement includes a selection of cages, tension band plates, and forks. A jig is used to place the holes in the tibial tuberosity at the proper angle and spacing, a fork inserter is used to insert the forks to secure the tension band plate into the bone, a T-handle with spreaders is used to bend the plates and to hold the osteotomy open for testing of the cage size and insertion of the cage implant. Pins are used to lock the jig in place and a plate bender is used to bend the plates and the ears of the cage.

The fork is inserted through the plate into drilled holes and the osteotomy is advanced. The amount of advancement is predetermined and the proper cage is selected and installed. The selected cage is placed approximately 4-6 mm below the joint surface and secured medially. The osteotomy is then closed over the cage and the distal most holes in the tension band plate are secured using screws. Once the two distal screws are placed in the plate, the resulting void is filled in with a graft.

Presently, the procedure is complex and expensive due to the need for the implanted cage, the tension plate, the fork, the jig, the fork inserter, the T-handle with spreaders, and the plate bender.

### BRIEF SUMMARY OF THE INVENTION

It is therefore an object of this invention to provide a new tibial tuberosity advancement implant.

It is a further object of this invention to provide such an implant which has fewer components.

It is a further object of this invention to provide such an implant which is easier to install.

It is a further object of this invention to provide such an implant which is strong.

It is a further object of this invention to provide such an implant which enables bone growth.

It is a further object of this invention to provide such an implant which is biocompatible and light weight.

It is a further object of this invention to provide such an implant which reduces the surgery time and the chance of infection.

It is a further object of this invention to provide such an implant which does not require numerous specialized pieces of instrumentation.

It is a further object of this invention to provide such an implant that allows increased flexibility in the amount of advancement.

It is a further object of this invention to provide such an implant that allows 2-17 mm of advancement at 1 mm increments.

It is a further object of this invention to provide such an implant with “locking wings” to mechanically fixate the osteotomy.

It is a further object of this invention to provide such an implant that contacts the inner cortical walls of the osteotomy and tibia once installed.

The subject invention results from the realization, in part, that a better tibial tuberosity advancement implant includes first and second arms in a V configuration and made of an open frame-like structure with anchor apertures extending inwardly from one arm across the other arm to maintain the spacing between the arms during the tibial tuberosity advancement procedure.

The subject invention features a tibial tuberosity advancement implant comprising first and second arms pivotally joined at an apex, tabs including anchor apertures extending from each arm for attachment of the arms in a transverse osteotomyized tibia and a selection of various with keys each insertable between the arms for setting the distance between the arms and the width between the osteotomyized tibia.

In one example, select tabs extend inwardly form one arm across the other arm to maintain the spacing between the arms. Typically, each arm includes multiple spaced channels for a key for varying the distance between the arms. One preferred key includes opposing convex members receivable in the channels. There is also typically a compression element extending between the opposing convex members.

In one embodiment, the arms taper in width from a narrow portion proximate the apex to a widest portion at the distal end of the arm. Also, each arm may include a framework construction to promote bone ingrowth about the implant. Preferably, the anchor apertures are chamfered.

One implant in accordance with the subject invention includes first and second arms pivotally jointed at an apex each including a plurality of spaced channels, anchor apertures extending from each arm for attachment of the arms, and at least one key insertable between the arms in the channels for setting the distance between the arms. In one example, there are a selection of varying width keys.

An implant kit in accordance with the subject invention features a plurality of different size implants each including first and second arms pivotally joined at an apex, each arm including a plurality of spaced channels, and anchor apertures for each arm for attachment of the arms. A plurality of different size keys are each insertable between the arms in the channels for setting the distance between the arms. The distance between the arms is then defined by the size of the implant, the size of the key, and the position of the key channels selected.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic three-dimension front view of an example of a tibial tuberosity advancement implant in accordance with the subject invention;

FIG. 2 is a schematic top view of the implant shown in FIG. 1;

FIG. 3 is a schematic front view of the implant shown in FIGS. 1 and 2 before the arms are bent into a V configuration;

FIG. 4 is a schematic front view showing a selection of three different width keys used to maintain the spacing between the arms of the implant;

FIG. 5 is a schematic front view showing one arm of the implant of the subject invention including a number of key receiving channels;

FIG. 6A-6C are views of the bottom of an implant in accordance with the subject invention and show how different width keys between the arms result in different arm spacings; and

FIG. 7 is a schematic side view showing a tibial tuberosity advancement procedure on the knee of a dog in accordance with the subject invention.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

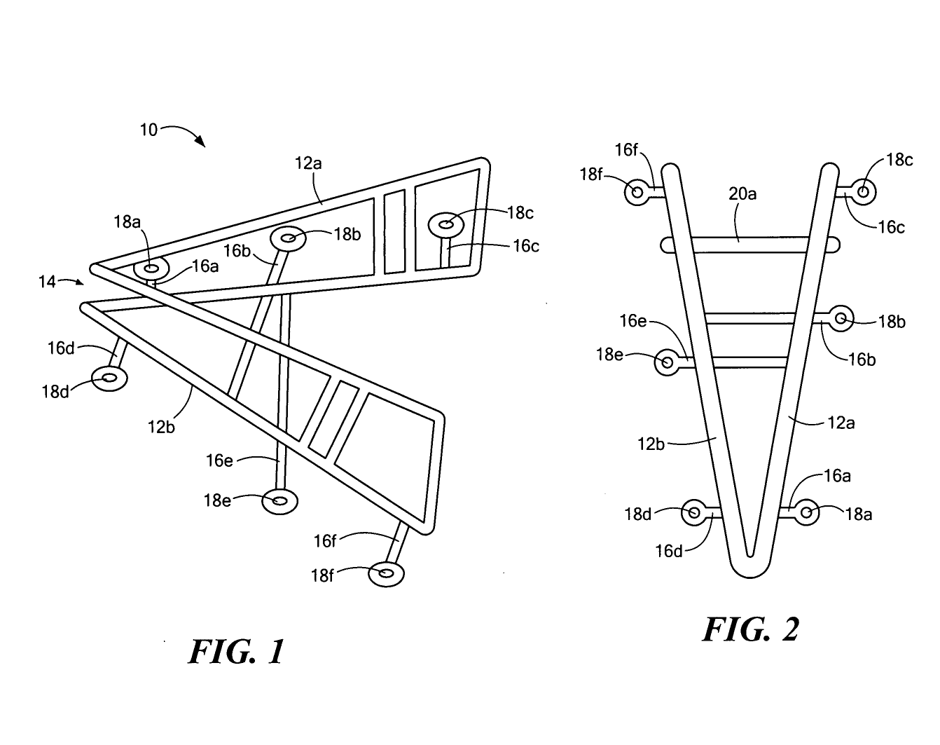

FIGS. 1-2 show an example of a tibial tuberosity advancement implant 10 in accordance with the subject invention. First and second arms 12a and 12b are pivotally joined at apex 14. Tabs 16a-16c extend outwardly from an edge of each arm 12a and tabs 16d-16f extend outwardly from arm 12b. Each tab terminates in an anchor aperture 18a-18f typically chamfered to allow the bone screws to be seated in the implant.

A key such as key 20a, FIG. 2 is insertable between arms 12a and 12b for setting the distance between the arms and the width of the osteotomyized tibia. A specially designed forceps can be used to insert the key. Anchor apertures 18a-18f attach the arms in the transverse osteotomyized tibia in a spacing as determined by the key selected. Typically, there is a selection of various width keys provided to the veterinary surgeon as well as various size implants. As shown in FIG. 2, intermediate tab 16b extends inwardly from arm 12b across and then outwardly from arm 12a and intermediate tab 16e extends inwardly from arm 12a and across arm 12b to maintain the spacing between the arms during the tibial tuberosity advancement procedure.

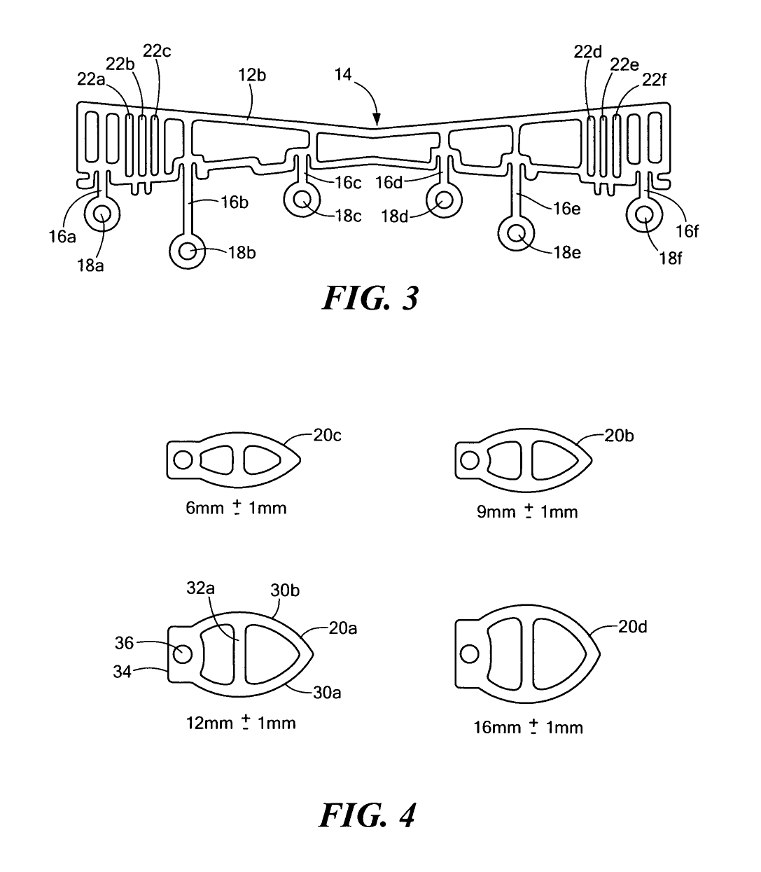

FIG. 3 shows arms 12a and 12b before they are bent or pivoted into a V-shape. Arm 12b includes channels 22a-22c and arm 12a includes corresponding opposing channels 22d-22f. In the V configuration, placing a key of a given width in channels 22c and 22d results in a further spacing of arms 12a and 12b than were the key place in channels 22a and 22f. In this way, the spacing between the arms can be varied by choosing where between the arms the key of a given width is placed. The spacing between the arms is further selectable by choosing different width keys. FIG. 4 shows four different width keys 20a-20d. Key 20b is sized in width to result in a spacing of arms 12a and 12b, FIG. 3, of 9 mm when placed in center channels 22b and 22e of each arm, and a spacing of 10 mm when placed in front channels 22c and 22d of arms 12a and 12d. A spacing of 8 mm results when key 20b is placed in rearward channels 22a and 22f of arms 12a and 12b.

Each key 20a, FIG. 4 in this particular example includes opposing convex members 30a and 30b receivable in the opposing channels in the arms. Compression element 32a extends between members 30a and 30b for strength. Tab 34 with opening 36 therein enables the insertion of the key between the arms of the implant during the surgical procedure.

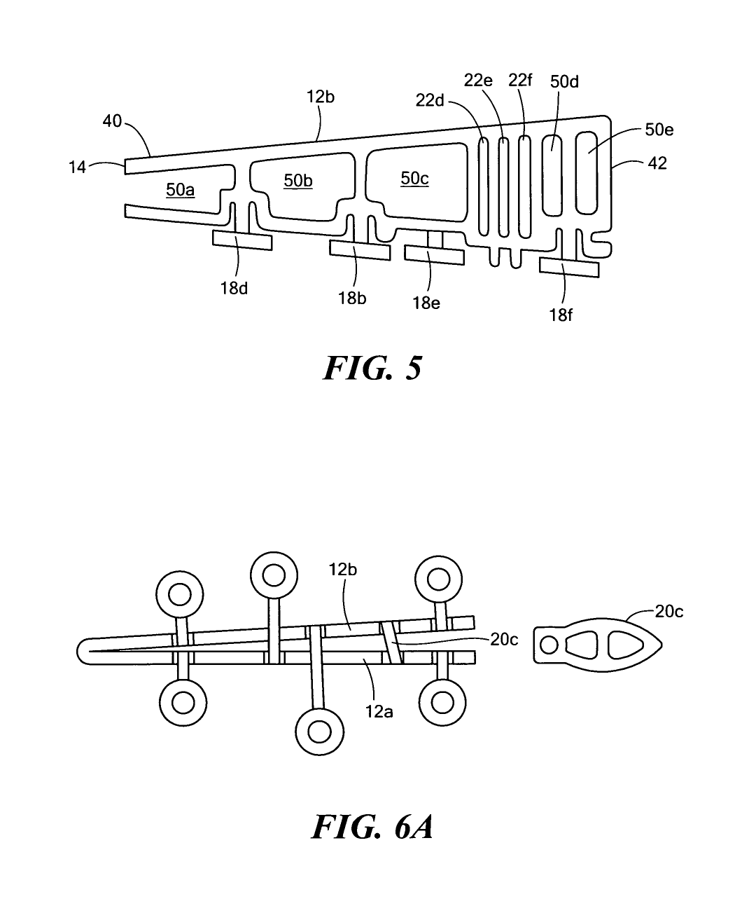

As shown in FIG. 5, each arm 12b preferably tapers in width from a narrow portion 40 proximate apex 14 to a wider portion 42 at the distal end of the arm. Thus, in the cranial/caudal view, the implant also forms a V. The geometry of this V shape ensures proper contact with the cortical bone and follows the contours of the canine tibia. Also, each arm 12b preferably includes a framework-like construction with open spaces 50a-50c to promote bone ingrowth about the implant.

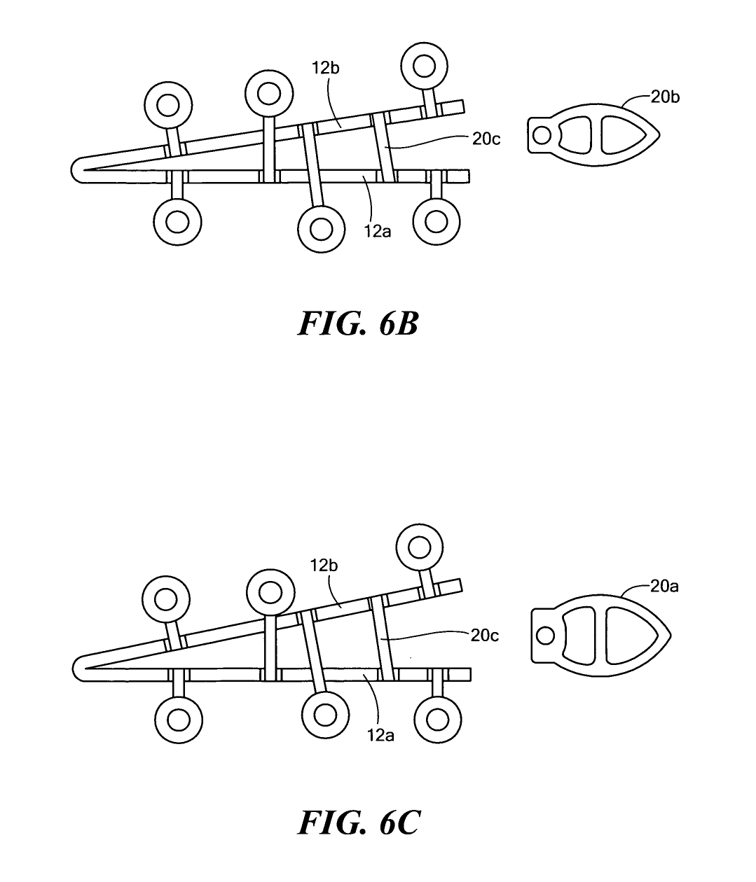

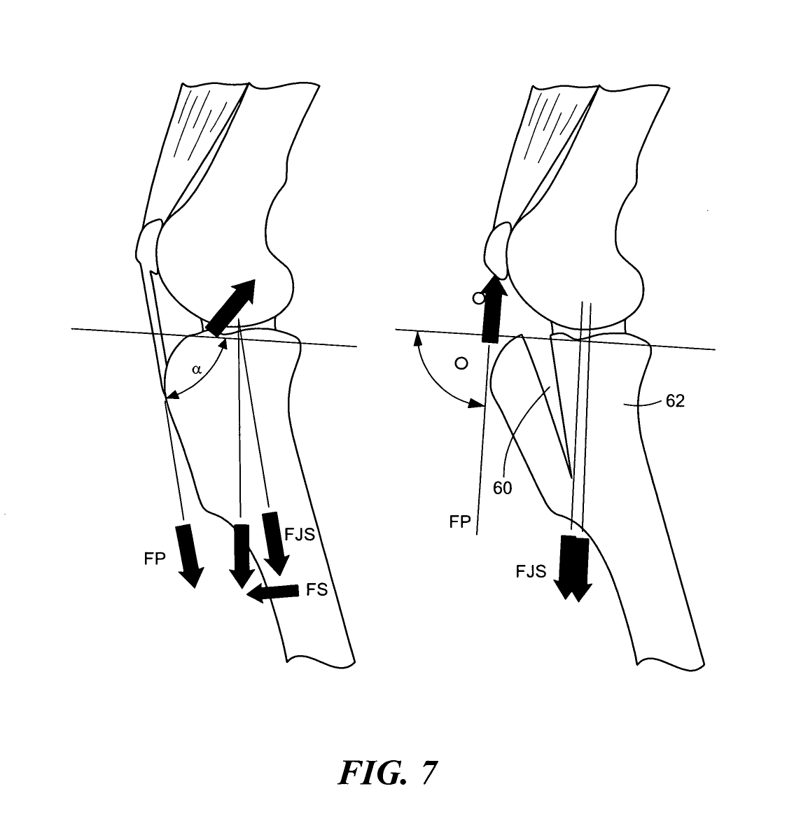

FIGS. 6A-6C depict how narrow key 20c results in a spacing of between 6 mm and 7 mm between arms 12a and 12b depending upon which channels are used while wider key 20b results in a spacing of 8 mm and 10 mm between arms 12a and 12b again depending upon which channels are selected. Key 20c results in a spacing of between 11 mm and 13 mm. A still wider key, 12d, results in an arm spacing of between 15 mm and 17 mm. With implants of different sizes, the use of different key channels, and different key widths results in the ability to accommodate a tibial tuberosity advancement procedure in dogs of widely varying sizes and allows the surgeon to properly choose the correct tuberosity advancement.

FIG. 7 shows transverse osteotomy 60 in the sagital plane of proximal tibia 62. The amount of advancement is determined prior to insertion of the implant. The measurement to determine the displacement is taken from the pre-operative x-ray. Once the cut is made in the tibial tuberosity, the spacer corresponding to the amount of advancement is inserted, medially, into the opening. The two distal and two proximal anchoring holes are secured using the appropriate bone screw. A bone reduction forceps is then used to clamp the osteotomy securely against the implant and the proximal tibia. Once secure, the middle (locking) anchoring holes are secured using the appropriate bone screws. At this point the osteotomy is fixated.

The result, in the preferred embodiment is a new tibial tuberosity advancement implant with fewer components. In any embodiment, the implant of the subject invention is easier to install, is strong, and preferably enables bone growth due to its overall construction. The preferred tibial tuberosity implant is also biocompatible and lightweight, typically made of titanium. One benefit of the subject invention is that the surgery time is reduced and thus the chance of infection is also reduced.

Typically, additional specialized instrumentation are not needed other than a tool used to insert the key between the V-arms. The implant of the subject invention has increased flexibility in the amount of arm displacement. In one example, the implant of the subject invention allows for 2, 3, 4, 5, 6, 7, 8, 9, 10, 11, 12, 13, 15, 16 and 17 mm spacings with fewer parts. Cross tabs 16b and 16e, FIG. 1 lock implant 10 in the proper orientation and maintain the proper angle between arms 12a and 12b. Proximal and distal tabs 16b, 16f, 16a, and 16c attach implant 10 at the bone and allow for placement of the chosen key without movement of the arms. Tabs 16a-16f are typically adjustable for proper screw placement in to the bone via anchor apertures 18a-18f. The design of the various keys allows for a simple insertion and a locking action between the arms and exhibits a resistance to being pulled out between arms 12a and 12b. In one example, there are a selection of three differently sized implants and four different sized keys.

Thus, although specific features of the invention are shown in some drawings and not in others, however, this is for convenience only as each feature may be combined with any or all of the other features in accordance with the invention. The implant described herein may also find uses for other medical procedures such as spinal spacing. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

Other embodiments will occur to those skilled in the art and are within the following claims.

What is claimed is:

1. A tibial tuberosity advancement implant comprising:
first and second arms pivotally joined at an apex;
tabs including anchor apertures extending from each arm for attachment of the arms in a transverse osteotomyized tibia; and
a selection of various width keys each insertable between the arms for setting the distance between the arms and the width of the osteotomyized tibia.

2. The implant of claim 1 in which select tabs extend inwardly from one arm across the other arm to maintain the spacing between the arms.

3. The implant of claim 1 in which each arm includes multiple spaced channels for a key for varying the distance between the arms.

4. The implant of claim 3 in which each key includes opposing convex members receivable in said channels.

5. The implant of claim 4 further including a compression element extending between the opposing convex members.

6. The implant of claim 1 in which each arm tapers in width from a narrow portion proximate the apex to a widest portion at the distal end of the arm.

7. The implant of claim 1 in which each arm includes a framework construction to promote bone ingrowth about the implant.

8. The implant of claim 1 in which the anchor apertures are chamfered.

9. An implant comprising:
first and second arms pivotally jointed at an apex each including a plurality of spaced channels;
anchor apertures extending from each arm for attachment of the arms; and
at least one key insertable between the arms in the channels for setting the distance between the arms.

10. The implant of claim 9 in which there are a selection of varying width keys.

11. The implant of claim 9 in which the anchor apertures are formed in tabs and select tabs extend inwardly from one arm across the other arm to lock the spacing between the arms.

12. The implant of claim 9 in which the key includes opposing convex members receivable in said channels.

13. The implant of claim 12 further including a compression element extending between the opposing convex members.

14. The implant of claim 9 in which each arm tapers in width from a narrow portion proximate the apex to a widest portion at the distal end of the arm.

15. The implant of claim 9 in which each arm includes a framework construction to promote bone ingrowth about the implant.

16. The implant of claim 9 in which the anchor apertures are chamfered.

17. An implant kit comprising:
a plurality of different size implants each including first and second arms pivotally joined at an apex, each arm including a plurality of spaced channels, and anchor apertures for each arm for attachment of the arms; and
a plurality of different size keys each insertable between the arms in the channels for setting the distance between the arms, the distance between the arms defined by the size of the implant, the size of the key, and the position of the key channels selected.
